SEQUENCE LISTING (2 sequences)

SEQ ID NO: 1 - protein, 15 aa, Artificial Sequence
  MOD_RES (1): FLUORESCEIN ISOTHIOCYANATE
AEEEIYGEFEAKKKK

SEQ ID NO: 2 - protein, 4 aa, Artificial Sequence
  MOD_RES (1): FLUORESCEIN ISOTHIOCYANATE
KKKK

(19) United States
(12) Patent Application Publication
(10) Pub. No.: US 2002/0112959 A1
(43) Pub. Date: Aug. 22, 2002

(54) Unbiased sample injection for microfluidic applications

(76) Inventors: Qifeng Xue, Fremont, CA (US); Ian Gibbons, Portola Valley, CA (US); Randy M. McCormick, Santa Clara, CA (US)

Correspondence Address: PERKINS COIE LLP, P.O. BOX 2168, MENLO PARK, CA, 94026, US

(21) Appl. No.: 09/972,709

(22) Filed: Oct. 4, 2001

Related U.S. Application Data
(60) Provisional application No. 60/239,018, filed on Oct. 4, 2000.

(51) Int. Cl. G01N 27/26; C12Q 1/68; G01N 27/447

(52) U.S. Cl. 204/453; 435/6; 204/604

(57) ABSTRACT

Methods, devices, and systems for unbiased transport of materials on a microfluidic device are disclosed, including methods of maintaining the starting composition of an analyte during transport, and methods of simultaneously analyzing both cationic and anionic components of an analyte. Analyte is loaded into a four-way junction of channels by controlled differential pressure applied to the channels. After analyte loading, an electrical potential is established, forcing charged species into at least one of two separation channels.

### This application claims priority to U.S. Provisional Application No. 60/239,018 filed Oct. 4, 2000, which is incorporated herein by reference in its entirety.

### BACKGROUND OF THE INVENTION

Microfluidic systems are becoming increasingly important for generation of chemical and biological information. In contrast to older separation technologies using separation channels in the range of millimeters to centimeters and handling samples with volumes of several microliters to multi-liters, microfluidic devices typically have channels and reservoirs that are dimensioned in micron-to-submicron ranges, and generally handle sample volumes in the range of microliters to nanoliters. Microfluidic systems are capable of generating information comparable to the quality of conventional systems, but are much faster and less expensive due to their smaller scale. Consequently, applications requiring the performance of very large numbers of relatively simple assays, such as genotyping or chemical screening, can benefit tremendously from use of their use.

A wide variety of applications have been adapted to a microfluidic scale. For example, U.S. Pat. No. 6,074,827, incorporated herein by reference, discloses a range of uses of microfluidic devices for electrophoretic applications, including clinical assays, high throughput screening for genomics and pharmaceutical applications, in vitro diagnostics, molecular genetic analysis, cell separations, and others. These various applications rely on fluid transport. A variety of mechanisms have been developed for controlling fluid movement on a miniaturized platform, and are generally adaptations of methods used in larger-volume systems. For example, some of the earlier devices made use of mechanical micropumps and valves (see for example, WO 98/52691, U.S. Pat. No. 5,997,263; U.S. Pat. No. 5,271,724; U.S. Pat. No. 5,375,979). However, their mechanical and operational complexities have limited their utility on a true microscale.

Perhaps the most common form of controlling material transport within a microfluidic device makes use of electric fields, either as electrophoretic forces that move charged molecules through a medium (see, e.g., U.S. Pat. No. 6,093,296 and U.S. Pat. No. 5,750,015) or electroosmotic forces that move fluid in bulk (Purnendu K. Dasgupta, etal. (1994) Electroosmosis: A Reliable Fluid Propulsion System for Flow Injection Analysis, Anal. Chem., 66:1792-1798). These electrokinetic forces provide modes of material transport that are very fast, relatively easy to devise, and allow fine levels of control. However, in some instances the use of electric fields results in a number of disadvantages, which have been collectively referred to as electrophoretic bias, described in some detail by Parce et al. in U.S. Pat. No. 6,042,709. This bias results from different species having different electrophoretic mobilities, which are affected by molecular weight and the amount and polarity of charge of a molecule. These differences in electrophoretic mobilities cause separation of the components of a mixture, resulting in a change in sample composition during the transport process. In addition, the components of interest in a sample may be diluted by electrophoretic transport when the sample contains excess salts. This dilution arises from salt ions carrying a significant fraction of the total current. Another source of bias results from movement of positively and negatively charged species in opposite directions. For these reasons, electrophoretic fluid movement does not allow a simple, unbiased fluid transport.

Some of the disadvantages of electrophoretic transport can be overcome by using electroosmotic forces for bulk transport of fluids. With this methodology, a microchannel has functional groups at its surface that ionize, creating a net surface charge opposite to that of solvent contained in the microchannel immediately adjacent to the surface. With the application of an electric field across the channel, charged molecules in the solvent adjacent to the channel surface will migrate to the appropriate electrode, causing a bulk drag of solvent within the microchannel. However, the use of electric fields to drive electroosmotic transport also results in some electrophoretic separation of components of the mixture within the volume of fluid being transported. Any application of an electric field will change the composition of a mixture locally, leading to an electrophoretic bias in the sample. Although methods and devices have been disclosed to compensate for this bias (see, e.g., U.S. Pat. No. 6,042,709), these processes are cumbersome, and only attempt to compensate for the bias rather than prevent it, leading to unpredictable results. Furthermore, the techniques are poorly suited to maintaining a mixture of cationic and anionic species, and they require introduction of salts into the system, complicating any subsequent manipulations of the sample. For manipulations in which it is necessary to maintain the starting composition of a mixture, the range of microfluidic applications currently available is very limited.

In addition to retaining the compositional profile of a mixture, it is often advantageous to avoid dilution and to control the shape of an injected sample. Both of these factors help preserve signal strength on a microfluidic device. When using such a device, the injected sample is generally created on the device itself in order to deliver a very small volume with minimal dilution and diffusion. This is usually accomplished by arranging overlapping channels to form an intersection (U.S. Pat. No. 6,007,690 and U.S. Pat. No. 5,770,029, both incorporated herein by reference). Sample is streamed through one of the channels across the intersection, and the contents of the intersection are then injected into the intersecting channel for separation. A method using electric fields for spatially focusing material traveling across an intersection of two microchannels is disclosed in U.S. Pat. No. 5,858,187, however, it subjects a sample to all of the aforementioned electrophoretic biases.

In summary, the present art is deficient in providing methods for moving reagents on a microfluidic device that does not bias the reagent, that are controllable, and that work with good reproducibility on a microfluidic scale.

### SUMMARY OF THE INVENTION

The present invention provides microfluidic methods and systems for electrophoretic separations that are capable of an unbiased transport of liquid samples within a microfluidic device. An unbiased transport of materials is achieved by establishing a pressure differential between liquids contained in the microchannels. The present invention also provides methods and devices for simultaneously characterizing positively and negatively charged species contained in a sample by using controlled differential pressure to transport and create an injection sample containing all charged species of the original analyte.

Certain embodiments disclose methods to control the shape of the injected sample while maintaining its composition. Material is transported by a pressure differential from the supply end to the waste end of a sample supply microchannel, passing through a junction with a separation microchannel. Sample material is spatially confined within the junction by streams of liquid emerging from arms of the separation microchannel flanking the junction, to form an injection sample stream in the junction with substantially the same composition as the original liquid sample. The confined injection sample is formed by creating a negative pressure on the waste end of the sample supply microchannel relative to that of the supply end of the sample supply microchannel and both ends of the separation microchannel.

Further embodiments of the present invention disclose coordinated systems for sample injection, separation, data collection, and analysis.

### BRIEF DESCRIPTION OF THE DRAWINGS

FIG. 1 is a diagrammatic view of an embodiment of a system for separation and detection of a sample according to the present invention. The double-headed arrow indicates the cross-sectional side view perspective of FIG. 2.

FIG. 2 is a diagrammatic cross-sectional view illustrating one embodiment of subcomponents of a system according to the invention for creating and controlling pressure on a microchannel contained in a microfluidic device.

FIG. 3(A, B) illustrates subcomponents of the microfluidic system suitable for use in the present invention. This system also includes devices for creating a controlled differential pressure over one or more fluid reservoirs, which is not shown in order to clarify details of other subcomponents of the system. FIG. 3A shows an embodiment of the system in which the microchannels of the microfluidic device intersect to form a simple cross. FIG. 3B shows another embodiment of the microfluidic device, with the two arms of the sample microchannel 21 offset from one another.

FIG. 4(A, B) are diagrammatic views of the shape of sample material streaming through the intersection of two microchannels. FIG. 4A illustrates the sample shape resulting from transport without focusing. Pressure is used to transport sample from the sample source microchannel 24 to the sample waste microchannel 25, while keeping the pressure differentials of the two arms of the separation microchannel 22 neutral relative to the junction 23. “High” and “low” indicate relative pressures exerted on the individual microchannel arms. FIG. 4B illustrates the sample shape resulting from focusing the sample while it is transported through the microchannel junction 23. The sample is focused by confining it within the junction with material streaming into the junction from the two arms of the separation microchannel 22.

FIG. 5 shows video images of sample focusing using a partial vacuum to control sample shape.

FIG. 6(A, B) contains electropherograms showing unidirectional separation profiles of a sample that was focused, injected, and separated by various methods. The starting points of multiple separation profiles presented in a single panel (such as “a,” “b” and “c” in panel 6A) are offset from one another to facilitate comparison of the profiles. FIG. 6A electropherograms were generated using various methods to transport sample and focus within the microchannel junction prior to separation. Peaks bracketed by the number 1 represent fluorescein; peaks bracketed by the number 2 represent FITC-peptide 1. The third small peak results from breakdown products of the two fluorescent species in the sample. Profiles:

a: sample transported across the microchannel junction electrokinetically without sample focusing, separation conducted without electrophoretic pullback;

b: sample transported across and focused within the microchannel junction electrokinetically, separation conducted with electrophoretic pullback;

c: sample transported across and focused within the microchannel junction with a partial vacuum; separation conducted with electrophoretic pullback.

FIG. 6B electropherograms were generated using a partial vacuum to transport sample and focus within the microchannel junction. Profiles:

b: separation conducted with electrophoretic pullback;

d: separation conducted without electrophoretic pullback.

FIG. 7(A, B) provides a diagrammatic view of two embodiments of methods according to the present invention for electrophoretic separation of sample constituents, wherein sample was transported to the microchannel junction 23 by differential pressure. FIG. 7A is an illustration of a unidirectional separation, using an electric field applied to all four reservoirs 28, 29, 30, and 31 to suppress migration of sample material into the separation microchannel after separation has started. FIG. 7B is an illustration of a bidirectional separation and detection.

FIG. 8(A, B) illustrates the impact of salt on the signal strength of separated species. Samples containing differing amounts of salt were focused by various methods for injection into a separation microchannel. The starting points of multiple separation profiles presented in a single panel are offset from one another to facilitate comparison of the profiles. FIG. 8A are electropherograms after electrokinetically focusing samples prior to injection. FIG. 8B are electropherograms after pneumatically focusing samples prior to injection.

FIG. 9 is an electropherogram showing a bidirectional separation profile of anions and cations in a sample after focusing the sample for injection using controlled differential pressure.

### DETAILED DESCRIPTION OF THE INVENTION

The present invention concerns methods and systems for transporting liquids in a microfluidic device without altering the composition of the liquid. This unbiased transport of materials is enabled by the use of controlled differential pressure, and allows a simultaneous separation and analysis of both cationic and anionic components of an analyte. In disclosing the various embodiments of the invention, the organization of the systems of the invention will first be described in general terms. Some representative embodiments of systems for using controlled differential pressure for unbiased transport will be disclosed in more detail, followed by more particular descriptions of the various subcomponents of the present systems, and examples of uses of these systems.

I. General Organization of a Microfluidic System

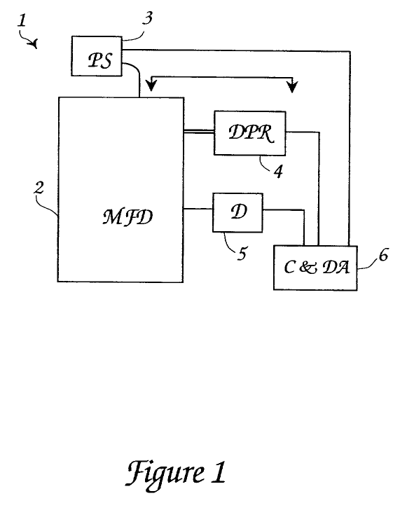

One embodiment of the configuration of devices contained in the systems of the present invention is shown in FIG. 1. The microfluidic system 1 includes a microfluidic device 2, a power supply 3, a differential pressure regulator 4, a detector 5, and a controller and data analyzer 6. The pressure regulator functions to regulate pressure on liquid samples contained in the microfluidic device, the power supply creates electrical potentials on the device for separations of molecular species within a sample, and the detector collects data given by the molecular species under analysis. A controller and data analyzer may optionally coordinate operation of the subcomponents of the device, and may also serve to receive, store, and analyze data generated by the system.

The subject systems enable an unbiased transport of sample material to a separation microchannel. By unbiased is meant that the composition and concentrations of molecular species within a sample are substantially unchanged (nominally the same) after transport of the sample. This unbiased transport is accomplished by creating pressure differentials between the contents of various microchannels in order to cause bulk movement of fluids in a controlled fashion. This unbiased material transport is unaffected by sample components that can impact electrophoretic mobilities, such as salts. These properties enable use of the present invention to conduct a simultaneous, bidirectional separation of both positively and negatively charged species contained in a mixture. Many different types of entities may be usefully analyzed on the device, including atoms, molecules, molecular assemblies or subassemblies, particles, organelles, whole cells, etc. Furthermore, complex mixtures of entities with normally unresolvable electrophoretic mobilities can be resolved and analyzed by modification of strategic subsets within the mixture in manners that affect mobility, or by use of combinations of separation strategies.

The microfluidic device of the subject invention is an electrophoretic microdevice fabricated as a single compact unit. By microfluidic is meant liquid or fluid materials with volumes consistent with those of the microchannels in a microfluidic device, typically in the range of microliters to nanoliters. The microfluidic device comprises at least a sample microchannel and a separation microchannel that intersect at a junction. These microchannels may intersect to form a simple cross, or one arm of the sample microchannel may be offset from the other arm on the opposite side of the junction formed with the separation microchannel. The sample supply microchannel will terminate at each end with a reservoir, and the separation microchannel may terminate at one or both ends with a reservoir. By microchannel is meant that the cavity within the microfluidic device in which liquid medium is present is a conduit, e.g., channel or cylinder, which may be either enclosed or open to atmosphere, which is present on the surface of or wholly contained within a planar substrate comprised of one or more layers, and that has a cross sectional area providing for capillary flow through the conduit. By reservoir is meant a cavity within the microfluidic device that is in fluid conducting relationship with at least one microchannel, which may be either enclosed by a chamber or open to atmosphere, that provides access for introduction of fluid materials and electrodes, and that is situated in the microfluidic device in a manner providing for control of the pressure exerted on the fluid materials contained therein.

The subject systems comprise a pressure regulator for generating and controlling pressure on liquid samples contained in the microfluidic device. By pressure is meant hydraulic or pneumatic pressure. Pressure may be exerted on the contents of the reservoirs or microchannels of the device, either singly or in combination, and may be exerted by creating a positive pressure, negative pressure, or a combination thereof. Pressure may be exerted on liquid material in the microfluidic device either directly or indirectly. Direct pressure may be exerted by, e.g., hydrostatic force, active fluid pumping within a closed chamber, or piston action within a closed chamber. Indirect pressure may be exerted via, e.g., an air gap, a diaphragm, or other arrangements.

The electrophoretic microdevices of the subject system are useful for separation of entities by the movement of those entities in a liquid medium under the force of an electrical field. Integral to the design of the present systems are devices for creating and controlling electrical potentials on the microfluidic device. Accordingly, electrodes are in contact with fluid contained in some or all of the reservoirs. Each electrode is operatively connected to a power supply, and is capable of being utilized independently from all other electrodes in the system, including the set voltage, grounding, floating, or time-dependent variations thereof.

The present system also comprises devices for detecting the species being analyzed. Any of a variety of signals associated with an entity of interest may be employed for detection, including fluorescent, luminescent, calorimetric, electrochemical, or radioisotopic. Fluorescent labels are preferred, including molecules such as fluorescein, rhodamine, pyrene, Cy5, Cy3, derivatives thereof, and the like. A separation microchannel within the microfluidic devices of the present system contains a region or regions having a transparent surface to allow transmission of signal from entities being separated in the microchannel to a detector operatively positioned in proximity to the transparent surface. The present system, comprising a microfluidic device, a pressure regulator, a power supply, and a detector, are integrally managed by a controller and data analyzer 6 that regulates the interactions of the various components, and which may also collect, control, and analyze the information produced by the system.

II. Establishing Controlled Differential Pressure

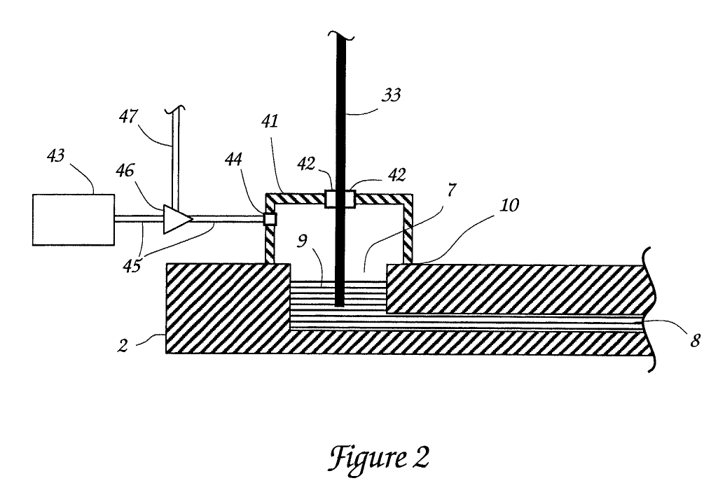

FIG. 2 illustrates one embodiment of an arrangement of subcomponents of the present system that enables establishment of controlled differential pressure on the contents of a microfluidic device. This figure provides a cross-sectional side view of the microfluidic device 2 from the edge (indicated by the double-headed arrow in FIG. 1), and includes several components of the differential pressure regulator 4 shown in FIG. 1. The microfluidic device 2 contains multiple microchannels, here exemplified by a single microchannel 8, which terminates with a reservoir 7. The microchannel is filled with fluid 9 introduced through the reservoir. Electrodes 33 connecting the microfluidic device to the power supply 3 (shown in FIG. 1), are placed in the reservoir in fluid conducting relationship with the fluid contained therein. Pressure on fluid contained in the reservoir and connecting microchannel is controlled by enclosing the space above the reservoir with a chamber 41, which forms a seal with the microfluidic device at the substrate surface 10 surrounding the reservoir, creating an enclosed space. The chamber incorporates an electrode seal 42 that maintains the closed space of the chamber while allowing entry of the electrode in order to contact fluid. A differential pressure supply 43 is operationally connected to the chamber by a connector 45, which joins the chamber through a pressure seal 44. The pressure established in the chamber is governed by a three-way valve 46, which controls connection of the differential pressure supply to the chamber, or opens the chamber to ambient pressure through a release line 47. The differential pressure supply 43 may be either a vacuum source to lower relative pressure or a pressure source to raise relative pressure. The differential pressure supply is linked to the controller and data analyzer 6 (shown in FIG. 1), which regulates connection of the differential pressure supply to the chamber through the course of sample loading, injection, and separation, and coordinates function of the differential pressure supply with other subcomponents of the system, including the power supply 3 and the detector 5.

Several devices are envisioned for establishing and controlling differential pressure. Methods suitable for creating differential pressure include preferably valveless pumps, or mechanical pumps. Valveless pumping methods suitable to the invention include forced air pressure, reduced pressure with a partial vacuum, gravity flow, centrifugation, and the like. Suitable mechanical pumps include a syringe, diaphragm pumps, peristaltic pumps, and the like. Preferred modes include vacuum or pressure applied to the contents of microchannels, either singly or in combination. The devices providing differential pressure force can be controlled manually, or programmed to function automatically. Flow rates can be monitored with internal standards in the course of using the device, providing feedback data to make flow rates more controllable and reproducible. Differences in pressure exerted on reservoirs to cause fluid movement are generally from about 0.001 psi to 1000 psi, usually from about 0.01 psi to 100 psi, and more usually from about 0.1 psi to 30 psi. Pressure differentials are generally imposed on fluids in a microfluidic device for about 1 millisecond to 30 minutes, usually from about 0.1 to 30 seconds, and more usually from about 5 to 15 seconds prior to initiating electrophoretic separations.

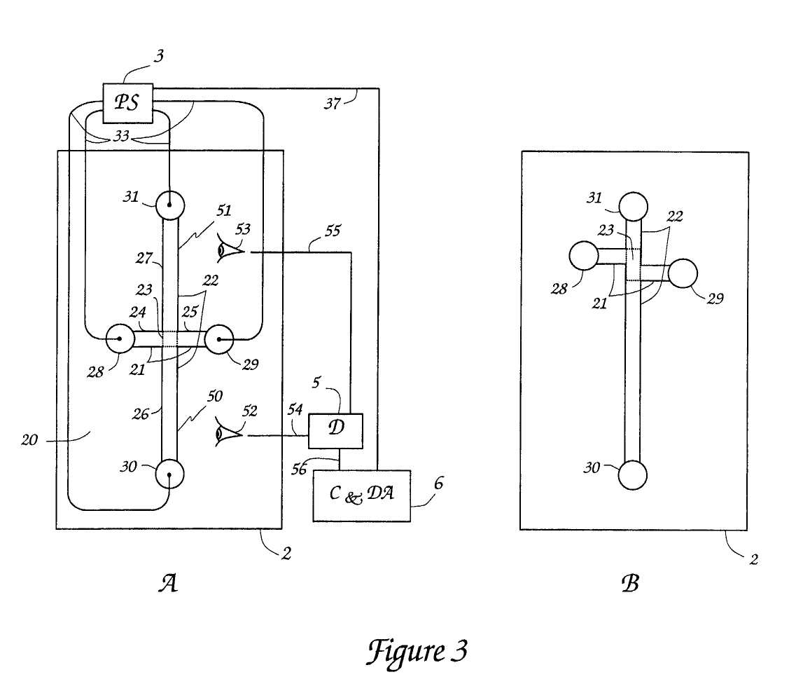

FIG. 3 provides a more detailed illustration of additional subcomponents of the present system. This system includes a microfluidic device 2 formed on a planar substrate 20, which may be fabricated from a wide variety of materials, including glass, metal, fused silica, plastics, and so forth. Preferred materials include thermoplastics, including polyacrylics, polynorbornenes, polycarbonates, polyolefins, and the like. Various components of the microfluidic device may be fabricated from the same or different materials, depending on the intended use of the device, economic concerns, solvent compatibility, optical clarity, auto-fluorescence, color, extrusion characteristics, mechanical strength, and the like. The devices may be fabricated using any convenient method, including conventional molding and casting techniques. The planar substrate of the device is manufactured to have microchannels and reservoirs disposed therein. A cover plate may then be sealed onto the surface of the substrate to create enclosed microchannels. A more detailed description of the manufacture of the subject microfluidic device is given in U.S. Pat. No. 5,110,514 and U.S. Pat. No. 6,074,827.

The microfluidic device, as depicted in FIGS. 3A and B, contains a sample microchannel 21 and a separation microchannel 22, which intersect at a microchannel junction 23, the boundaries of which are shown as dofted lines. The microchannels may have a variety of configurations, including linear, curved, angled, and may form one or more intersections with each other or with additional microchannels or elements. The intersections of these microchannels may be in the form of a simple cross, as illustrated in FIG. 3A, or one arm of the sample microchannel may be offset from the other arm on opposite sides of the junction formed with the separation microchannel, as illustrated in FIG. 3B. The spacing between the two arms and the cross-sectional area of the separation microchannel in the junction will define the volume and quantity of the injected sample. Microchannel arms arranged in an offset will generally be separated (center to center) by from about 20 μm to 5000 μm, usually from about 50 μm to 1000 μm, and more usually from about 100 μm to 500 μm. By arm is intended a portion of the length of a microchannel bounded by design elements such as a reservoir or a junction. The angles formed by intersecting microchannels may be of any convenient angle, most commonly a 90° angle. The cross-sectional shape of a channel may be circular, ellipsoid, rectangular, triangular, and so forth, forming a microchannel at the surface of the planar substrate in which it is present. The microchannel will have a cross-sectional area providing for capillary fluid flow through the microchannel, wherein the cross-sectional dimensions of width and height will be in the range of from about 1 μm to 200 μm, usually from about 10 μm to 100 μm, more usually from about 30 μm to 80 μm.

It is contemplated that reproducibility and the range of control of fluid flow can be refined through variations in the design of the microchannels. The dimensions of the microchannels, including cross-sectional area and shape, can be varied either within a microchannel or between two or more microchannels, in order to modify the degree of sample focusing or effect changes in pressure differentials between the channels to control rates of flow. The cross-sectional dimensions are more preferably changed by altering the width of a microchannel. Variations in microchannel width to alter pressure on liquids contained therein range from about 1 μm to 2 mm, usually from about 10 μm to 300 μm, and more usually from about 50 μm to 80 μm. Pressure can also be controlled by varying the height of liquids contained in the reservoirs.

The lengths of the microchannels may also be varied according to the application. The lengths of the arms of the separation microchannel are generally chosen based on the separation being conducted. For example, when a small number of species with large mobility differences are being separated, a short separation microchannel arm is desirable. When the sample comprises a large number of species, or species that are difficult to resolve, a longer separation microchannel may be required. For separations of 1 to 25 species, the length of an arm of a separation microchannel ranges from about 3 mm to 3 cm; for separations of 15 to 50 species, the length ranges from about 1 cm to 5 cm; for separations of 30 to several hundred species or more, the length ranges from about 5 cm to 50 cm.

In the embodiments illustrated in FIG. 3, two arms of the sample microchannel 21 are formed by intersection with the separation microchannel 22. A first arm, the sample supply microchannel 24, terminates at a first reservoir, also called the sample supply reservoir 28, where sample is introduced to the device. A second arm of the sample microchannel 21 on the opposite side of the microchannel junction 23 is a sample waste microchannel 25, which terminates at a second reservoir, also called the sample waste reservoir 29. The separation microchannel 22 comprises two arms on either side of the junction 23, including the first separation microchannel 26 and the second separation microchannel 27. The first separation arm terminates at a fourth reservoir, also called the first separation microchannel reservoir 30. The second separation arm terminates at a third reservoir, also called the second separation microchannel reservoir 31. Each reservoir may be open to atmosphere or enclosed to control pressure. Depending on the particular application and the nature of the materials being analyzed, one or more detection regions for detecting the presence of distinct species migrating through a microchannel is present in association with at least the separation microchannel 22. FIG. 3A discloses a first separation microchannel detection region 50 in association with the first separation microchannel 26, and a second separation microchannel detection region 51 associated with the second separation microchannel 27. Additional detection regions optionally may be incorporated, either within the separation microchannel, or elsewhere in the device as required. A detection region comprises a surface that allows transmission of signal from entities in the microchannel to a sensor operatively connected to a detector. The detection region will be fabricated from a material that is optically transparent, generally allowing transmission of light with wavelengths ranging from about 180 to 1500 nm, usually about 220 to 800 nm, and more usually about 250 to 800 nm. Suitable materials include fused silica, plastics, quartz glass, and the like. First and second separation microchannel sensors 52 and 53 are arranged in functional proximity to the transparent surfaces of the first and second separation microchannel detection regions, respectively. A detector 5 controls and collects data from the sensors through connections 54 and 55 between the detector and the first and second separation microchannel sensors, respectively.

In some separation applications, the voltage may be regulated across all of the microchannels within the microfluidic device to provide material transport. Accordingly, a microchannel can function as an electrophoretic flowpath, and will have associated with it at least one pair of electrodes for applying an electrical field to media present in the flowpath. Where a single pair of electrodes is employed with a microchannel, typically one member of the pair will be present at each end of the microchannel, most usually within reservoirs at the termini of the microchannel. Where applicable, a plurality of electrodes may be associated with the electrophoretic flowpath, as described in U.S. Pat. No. 5,126,022, the disclosure of which is herein incorporated by reference in its entirety, which can provide for precise movement of entities along the electrophoretic flowpath. In the embodiments illustrated in FIG. 3, both the sample microchannel 21 and the separation microchannel 22 function as electrophoretic flowpaths, each having associated electrodes 33, which are operatively connected to media present in the sample supply and sample waste reservoirs 28 and 29. Similarly, electrodes 33 are operatively connected to media present in the first and second separation microchannel reservoirs 30 and 31. Each of the electrodes of the device is capable of being controlled independently from all other electrodes. A power supply 3 connected to each electrode regulates the electric fields created by the electrodes within the various electrophoretic flowpaths.

The present system is coordinated and regulated by a controller and data analyzer 6. The controller and data analyzer has a connection 56 with the detector to regulate the detector's operational parameters and to collect data gathered by the sensors. The controller and data analyzer additionally can have a connection 37 with the power supply to regulate electric fields established through the electrodes. The controller and data analyzer also may be operationally connected to the differential pressure regulator 4 (not shown). Through all of these connections, the controller and data analyzer can serve to integrate the separate subcomponents of the system, functioning both to regulate each of the devices in the course of operation, and to collect, store, manipulate, and analyze data generated by the system.

III. Unbiased Sample Transport and Spatial Confinement

Miniature devices designed for capillary fluid flow require methods and devices for movement of small volumes of material. As with all sample handling and separation methods, miniaturized devices yield more optimal results when the volume and shape of the sample of interest can be precisely and reproducibly controlled. In particular, detection sensitivity and the degree of resolution of species contained in a mixture depend upon spatial confinement of the sample during transport. By spatial confinement is intended that a liquid material is manipulated in a manner that controls the shape and volume of the liquid material, wherein controlling includes either maintaining or changing. One manner in which the shape of a liquid sample can be spatially confined is by imposing boundaries on the sample with other fluids. Fluids can be caused to move by exerting a force on them, and they can be moved in bulk by using pressure as the force.

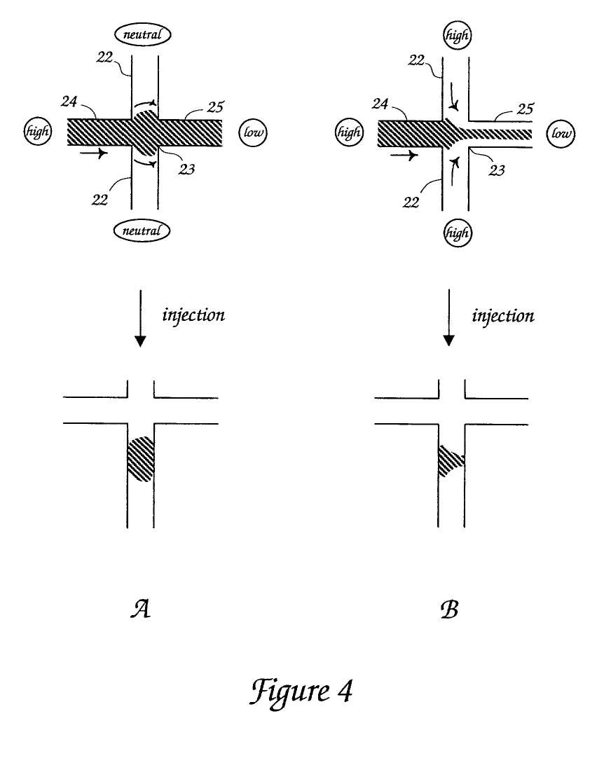

FIG. 4 illustrates the shape of a liquid sample moving through an intersection of two microchannels of a microfluidic device. By analogy to the device of FIG. 3, sample material flows from the sample source microchannel 24 on the left towards the sample waste microchannel 25 on the right, passing through the microchannel junction 23 formed by intersection with the separation microchannel 22. In FIG. 4A, sample is driven from the sample source microchannel to the sample waste microchannel by creating a difference in pressure between these two channels, while keeping both arms of the separation microchannel neutral by sealing them to prevent any pressure change relative to the junction. (Relative pressures on the sample source microchannel and sample waste microchannel are indicated as “high” and “low” in the figure. Arrows indicate the direction of flow.) Because the forces from the two arms of the separation microchannel 22 are neutral, sample passing through the junction will expand into the arms of the separation microchannel. This broadening increases the volume occupied by components of the sample, causing them to be diluted. When charged molecules from this sample stream are electrophoretically injected into the separation microchannel (as illustrated in the lower portion of FIG. 4A), components of the sample will form a broader band as a result of the broadening in the junction, making them more difficult to detect and more poorly resolved from one another.

These deficiencies can be avoided by spatially confining the sample as it passes through the junction of the microchannels. In FIG. 4B, sample again is flowing from the sample source microchannel 24 on the left towards the sample waste microchannel 25 on the right as a result of a pressure differential. However, in this scenario pressure between both arms of the separation microchannel 22 are increased relative to the sample waste microchannel 25, causing the sample stream to be spatially confined as it passes through the intersection. (As in FIG. 4A, the relative pressures between all four microchannel arms are indicated as “high” and “low,” and the arrows indicate the direction of flow.) When charged molecules within this sample stream are injected into the separation microchannel, components will be more readily detected because the sample has not been diluted by spreading in the junction. Furthermore, individual species will be better resolved because narrowing the sample will reduce the length of migration required for separation.

The system disclosed in FIG. 3 finds use in creating a spatially confined sample stream within a channel intersection, analogous to that illustrated in FIG. 4B. Various systems are envisioned for creating pressure differentials that provide unbiased liquid transport and spatial confinement within a microchannel junction. One embodiment incorporates a vacuum to reduce pressure on up to three microchannel arms. For example, the pressure on the sample waste microchannel 25 can be reduced relative to the other three microchannels via the sample waste reservoir 29 that will be enclosed in a chamber 41 connected to a vacuum supply (as illustrated in FIG. 2) to produce the relative pressure differences indicated as “high” and “low” in FIG. 4B. The remaining reservoirs can optionally also be enclosed in chambers to control their pressures relative to the sample waste reservoir, or simply be left open to ambient pressure. Another embodiment employs a pressure supply to increase pressure on up to three microchannel arms. For example, pressure is increased on the sample source microchannel 24 and both arms of the separation microchannel 22 via their respective reservoirs, each enclosed in a chamber. A pressure supply is utilized to increase pressure on these three reservoirs, creating the pressure differentials indicated as “high” and “low” in FIG. 4B.

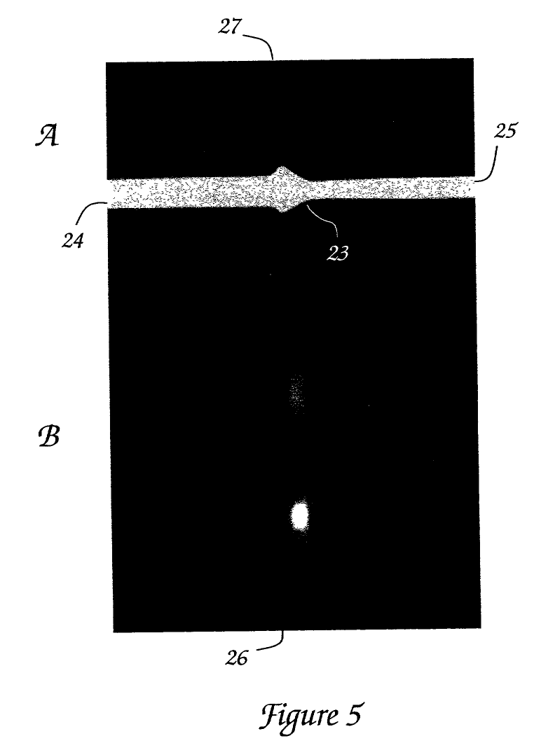

A photographic image of sample focusing within a microchannel intersection using a pressure differential is shown in FIG. 5A, which was created as described in Example 1. In this experiment, a sample containing two different fluorescent species was introduced into the sample supply reservoir at the terminus of the sample source microchannel 24. Differential pressure was created on the sample to cause it to flow through the microchannel junction 23 towards the sample waste microchannel 25, while being spatially confined by a simultaneous fluid flow from liquid contained in the first and second separation microchannels 26, 27. In this example, the differential pressure was created by establishing a partial vacuum at the terminus of the sample waste microchannel, while keeping the termini of the other three microchannels open to atmospheric pressure. This pressure differential was used to create a confined sample stream within the microchannel junction flowing from the sample source microchannel 24 to the sample waste microchannel 25. Once a confined sample stream is established within a microchannel junction, charged molecules within that sample stream can be injected into one or both separation microchannels 26, 27 for separation and detection of the components of the mixture. A photographic image of such a separation is shown in FIG. 5B. This image shows two bright spots corresponding to the two fluorescent components of the sample resolved from one another in the first separation microchannel 26.

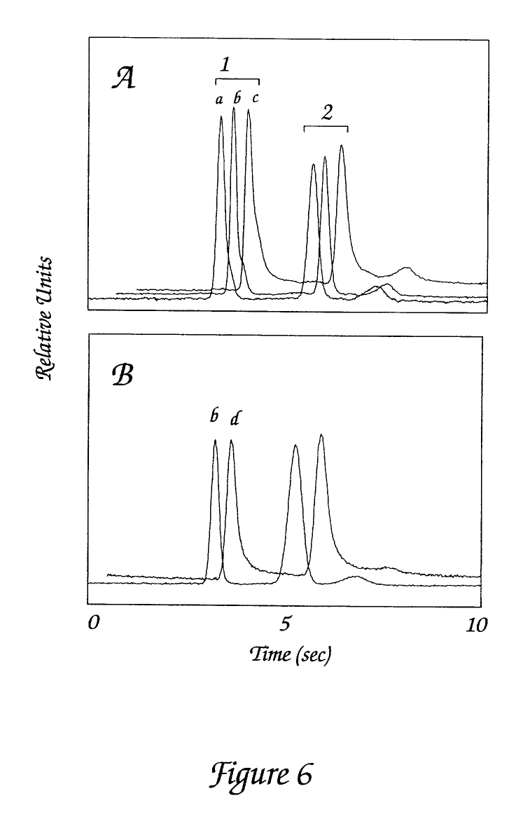

The quality of sample resolution using various methods to control the shape of the injected sample was assessed in a series of experiments presented in FIG. 6, and are described in detail in Example 2. When the individual species of an analyte are separated on a microfluidic device such as that shown in FIG. 3, the electrical potentials created on the four converging microchannels during separation may be established to pull uninjected charged sample material back into the sample supply microchannel 24 and the sample waste microchannel 25 in order to prevent a continuous flow of charged sample species from entering the first separation microchannel 26 after injection. To provide this sample pullback, the electrical potentials established on the sample supply and sample waste microchannels are generally set at an intermediate value relative to those of the first and second separation microchannels. This will cause one class of ions (the cations or anions, depending on the polarity of the electrical potential) to migrate from the second separation microchannel 27 into the other three microchannels, providing the pullback effect. The other class of ions will flow in the opposite direction from the sample supply and waste microchannels 24, 25 and the first separation microchannel 26 into the second separation microchannel 27. Use of pullback during separation provides a method of controlling the sample shape once it has entered the separation microchannel. FIG. 6A shows the results after sample was shaped for injection by various methods. Profile “a” is an electropherogram showing separation of the components of a sample shaped for injection by electrophoretic focusing, while with profile “b,” the sample was shaped by pneumatic focusing using a partial vacuum. For both of these experiments, subsequent separation was conducted with electrophoretic pullback. Profile “c” is a separation profile of sample that was not focused prior to injection, with the subsequent separation conducted without electrophoretic pullback. Comparison of profiles “a” and “b” shows that electrophoretic and pneumatic methods of shaping sample for injection yield similar results. Peak widths relative to height are very similar, and signals drop to background after a peak of material has passed the detector. Both methods of sample focusing contribute to improved resolution of distinct species in a separation. The improvement provided by sample focusing prior to injection can be seen by comparison to profile “c”. The peaks in this electropherogram are broader, and the baseline signal does not drop to a background level between the separated peaks. Both of these effects result from sample injection over the course of separation due to the lack of sample pullback.

The effect of sample pullback on separation is illustrated in FIG. 6B. The samples for both profiles “b” and “d” were shaped for injection by pneumatic focusing using a partial vacuum. The separation in profile “b” was conducted with sample pullback, while that of profile “d” was conducted without pullback. Comparison of these two electropherograms again shows that pullback during separation improves peak resolution by narrowing the peaks, and allowing signal to drop to background level between peaks. These results illustrate that pneumatic sample shaping prior to injection yields separation results comparable to electrophoretic focusing.

IV. Unbiased Sample Injection and Separation

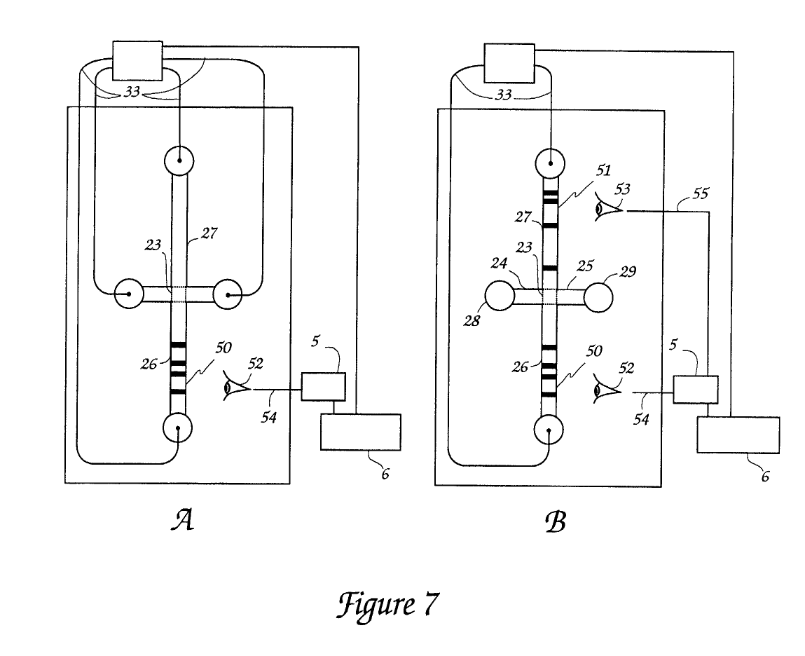

FIG. 7A illustrates operation of a system according to the present invention during a typical unidirectional electrophoretic separation. After transport of a sample mixture to a microchannel junction 23 and injection into the first separation microchannel 26, electrophoretic separation of either the anions or the cations within the sample is carried out with pullback, using all four electrodes 33 during the separation. The different species in the mixture will separate according to their electrophoretic mobilities, shown as separate bands in the figure. As components of the sample migrate down the separation microchannel, each will pass through a first separation microchannel detection region 50, which allows transmission of signal from the sample to the first separation microchannel sensor 52. The signal gathered by the sensor is transmitted via a connection 54 to the detector 5, which in turn delivers this information to the controller and data analyzer 6. When using pullback voltage settings, essentially no sample material enters the first separation microchannel 26 after injection, narrowing the peaks and allowing signal to drop to a baseline level, as shown in FIG. 6B. However, oppositely charged species are migrating into the second separation microchannel 36 from the other three microchannel arms, creating very broad signal peaks from the continuous flow of charged sample species from the sample supply and waste microchannels into the second separation microchannel through the course of electrophoresis. For this reason, the increased resolution obtained by using pullback conditions generally precludes monitoring separation of the oppositely charged species in the second separation microchannel 27.

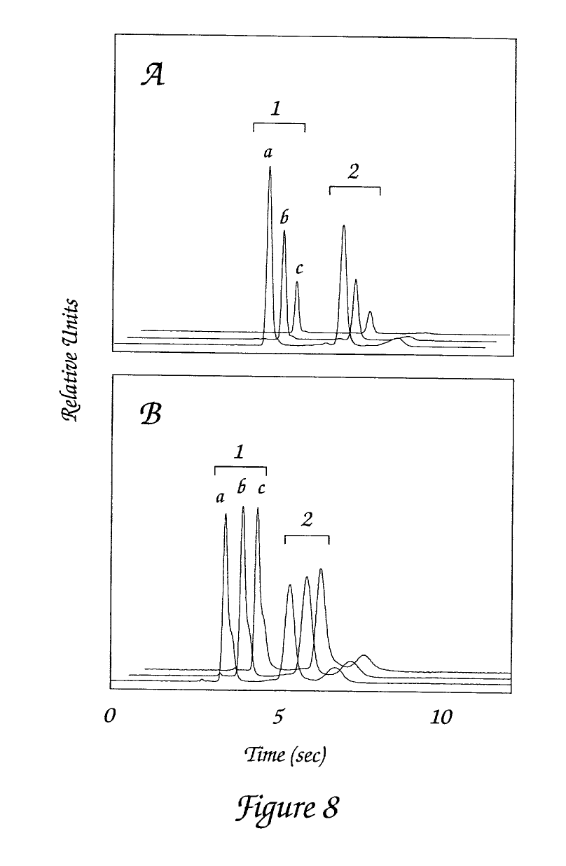

One source of sample bias caused by electrophoretic movement of fluids results from salts contained in the sample being analyzed. Because salt ions are small, and their concentrations in a reaction are often much higher than the analytes of interest, they will carry a significant fraction of the current in an electric field. As a result, other components in a sample will exhibit reduced mobilities relative to what they would have in a solvent with less salt. Furthermore, this electrophoretic bias increases as the relative mobility of a particular species is slower. Consequently, when samples are delivered electrophoretically to a position for injection in a separation microchannel, the concentration of the analytes of interest can be reduced dramatically, and this bias varies in proportion to a molecule's relative mobility. This type of electrophoretic bias is illustrated in the experiments presented in FIG. 8 (described in more detail in Example 3). A series of separations were conducted using samples containing two fluorescent analytes in a buffered solution with varying concentrations of NaCl. FIG. 8A shows separations conducted using electrophoretic focusing to shape the sample prior to injection in a microchannel for separation. Profile “a” shows sample with no NaCl, profile “b” is sample with 25 mM NaCl added, and profile “c” is sample with 50 mM NaCl added. The addition of salt dramatically reduces the amount of sample injected in the separation microchannel. By contrast, FIG. 8B shows the results with samples that were focused pneumatically prior to injection into a separation microchannel. Profile “a” was obtained from sample with no additional salt, profile “b” resulted from sample containing 25 mM NaCl, and profile “c” from sample containing 50 mM NaCl. Varying the amount of salt in the sample solution had a negligible impact on the concentration of sample injected into the separation microchannel after pneumatic focusing. The reduction in signal strength after electrophoretic focusing as compared to pneumatic focusing averaged about 55% with 25 mM NaCl, and about 75% with 50 mM NaCl.

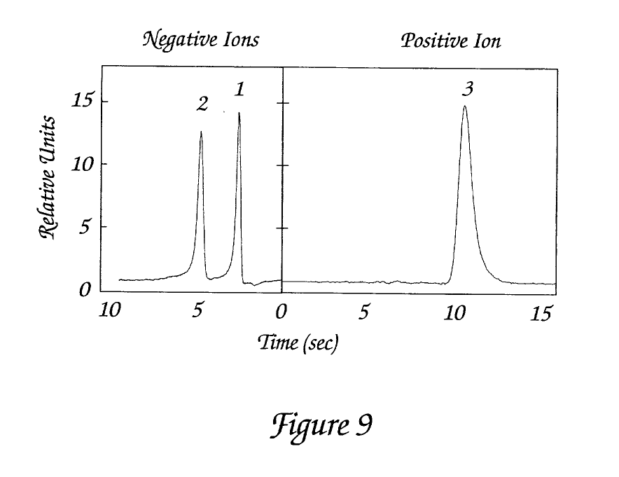

Because sample material is transported for injection into the separation microchannels without altering the composition of the sample, it is possible to separate both cationic and anionic species simultaneously by conducting a bidirectional separation. One embodiment of such a separation is illustrated in FIG. 7B. In this scenario, sample is transported to and focused within the microchannel junction 23 using differential pressure. Separation is conducted by creating a voltage gradient only between the first and second separation microchannels 26, 27, indicated by the presence of only two of the electrodes 33. The sample supply and sample waste reservoirs 28, 29 are allowed to float. No pullback is provided under these conditions, however the amount of charged species entering the separation microchannels 26, 27 is minimal because no electrical potential is established between them and the sample supply or sample waste microchannels 24, 25. After injection in the separation microchannels, a sample mixture comprising both cationic and anionic species will segregate in both directions, with cations migrating and separating along one separation channel, and anions migrating and separating along the other. Each class of charged species will pass through either the first separation microchannel detection region 50 or the second separation microchannel detection region 51, transmitting signal to the first separation microchannel sensor 52 or the second separation microchannel sensor 53, respectively. Both sensors transmit data via connections 54, 55 to the detector 5, which in turn delivers this information to the controller and data analyzer 6. Bidirectional separations are not possible with conventional electrophoretic systems because materials transported to the site of injection will inherently contain only cations or anions, depending on the electrical potential applied during focusing. Thus, the sample has been fractionated prior to delivery to the separation microchannels. An example of a bidirectional separation conducted on a sample that was delivered to the site of injection and focused using differential pressure is shown in FIG. 9, described in more detail in Example 4. Components of the sample injected included two fluorescent anionic species and one fluorescent cationic species.

When separations are conducted on a microfluidic device such as that shown in FIG. 3, pullback potentials are often created into the sample supply microchannel 24 and the sample waste microchannel 25 to prevent a continuous flow of charged sample species from entering the first separation microchannel 26 after injection. However, this pullback prevents simultaneous separation of both anionic and cationic sample species because it can only pull back one class of ions at a time. That is, if voltages are set such that anions flow from the second separation microchannel reservoir 31 to the sample supply, sample waste, and first separation microchannel reservoirs 28, 29, 30 (as with pullback), then cations will have the opposite migration pattern, and will continuously flow from the sample supply microchannel 24 and the sample waste microchannel 25 into the second separation microchannel 27. Using differential pressure, an unbiased sample containing both anionic and cationic species can be transported to the microchannel junction prior to separation. A simultaneous, bidirectional separation can then be conducted by using electrical potentials that do not provide pullback. This simultaneous bidirectional separation is not possible with conventional electrophoretic focusing because only the anionic or the cationic species contained in a sample will be transported to the microchannel junction, while the oppositely charged species will migrate to the electrode in the sample supply reservoir.

The following examples further detail possible embodiments of the invention. They are offered by way of illustration and not by way of limitation.

### EXAMPLES

A microfluidic system was created and used to demonstrate the utility of microfluidic sample delivery using differential pressure for reducing electrophoretic bias and for conducting bidirectional separations. The experiments described in the Examples were conducted with common parameters, as follows. Distinctive details are given with the Examples. The subcomponents of the system used in the Examples are diagrammed in FIG. 1, and include a microfluidic device, or card 2, which is operatively connected to a power supply 3 and a differential pressure regulator 4, both of which are connected to and controlled by a controller and data analyzer 6. Cards configured as shown in FIG. 3 are used for separation of organic analytes in an aqueous sample. The power supply creates appropriate electric fields on fluid samples in microchannels 24, 25, 26, 27 through independently controlled electrodes 33 introduced into each reservoir 28, 29, 30, 31 of the card. The differential pressure regulator creates and controls the air pressure above one or more of the reservoirs. The system also contains one or two sensors 52, 53 for collecting data for analysis of various characteristics of the injected sample. The sensors are operatively connected to a detector 5, which in turn is connected to the controller and data analyzer. Each sensor is positioned in proximity to detection regions 50, 51, in the first and second separation microchannels 26, 27 respectively, of the microfluidic device. Cards used in this system were fabricated from plastic, and contain microfluidic channels that are 80 μm wide and 30 μm deep. Data was collected using a custom built confocal microscope system equipped with a mercury lamp optical system and a Hamamatsu photomultiplier tube set at 600V.

The cards used for separations after electrokinetic sample focusing had the following channel lengths: sample supply, sample waste, and second separation microchannels, 5 mm; first separation microchannel, 10 mm. Cards used for separations after pneumatic focusing had the following channel lengths: sample supply microchannel, 5 mm; sample waste microchannel, 45 mm; first and second separation microchannels, 10 mm. With both cards, the detection regions are positioned 5 mm from the microchannel junction 23. Because of the different microchannel lengths, the voltages applied to the four reservoirs were adjusted to establish equal field strengths on the analogous microchannels between each microfluidic device. As a result, the different lengths had no impact on separation or peak shape.

To conduct a separation, all the reservoirs of a card except the sample supply reservoir are filled with separation buffer composed of 25 mM HEPES with 1% PEO, pH 7.4, and pulled to the sample supply reservoir to fill the microchannels. Samples used for separation experiments included 1 μM fluorescein and 3 μM FITC-peptide 1 (FITC-AEEEIYGEFEAKKKK, SEQ ID NO:1) (both anionic species), and in some experiments 3 μM FITC-peptide 2 (FITC-KKKK, SEQ ID NO:2) (a cation), contained in 50 mM HEPES with various concentrations of NaCl as indicated. 10 μL of sample was loaded into the sample supply reservoir 28. Electrodes were introduced into each of the four reservoirs, and voltages were independently applied as described in each experiment.

### Example 1

Photographic Imaging of Analyte Mixture Focused by Vacuum Differential

The microchannels of the device were filled with separation buffer, as described in the general protocol preceding this Example. Sample containing fluorescein and FITC-peptide 1 was introduced into the sample supply reservoir 28. A vacuum of 4 psi was applied to the sample waste reservoir 29 for 10 seconds, while keeping the sample supply reservoir 28, and the first and second separation microchannel reservoirs 30 and 31 open to atmosphere. Injection of sample material into the first separation microchannel 26 was done by breaking the vacuum, replenishing the sample waste reservoir by adding 8 μL separation buffer, and applying voltages to electrodes in each of the four reservoirs as follows: sample supply reservoir, 800V, sample waste reservoir, 1400 V, first separation microchannel reservoir 30, 1000 V, and second separation microchannel reservoir 31, 0 V. Separation was conducted for 20 seconds, while a confocal microscope equipped with a CCD camera collected images of the fluorescent analytes. FIG. 5A shows a photographic image of the confined sample stream prior to injection into the first separation microchannel microchannel. The unfractionated fluorescent sample appears white in this image, and is being constricted by fluids flowing from the first and second separation microchannels 26 and 27, as sample passes through the microchannel junction and into the sample waste microchannel. FIG. 5B illustrates the separation of the two fluorescent analytes at 4.5 mm from the point of injection. Lines have been drawn to highlight the borders and junction of the microchannels. Note that FIGS. 5A and 5B are separate images from different times in the experiment, brought together to better illustrate the shapes of the sample during creation of a confined sample stream and after an injection and separation. A complete image of the microfluidic device during the experiment would show material only as in FIG. 5A or 5B, and the two spots of FIG. 5B would be much further from the microchannel junction 23.

### Example 2

Resolution of Analyte Species Using Different Focusing Strategies

The effect of using different methods for sample formation within the microchannel junction 23 prior to injection into a separation channel was assessed by comparing resolution of two fluorescent species contained in a sample. The microchannels of the device were filled with separation buffer, as described in the general protocol preceding Example 1. Sample containing fluorescein and FITC-peptide 1 was introduced to the sample supply reservoir 28. In a first experiment, sample was transported to and focused within the microchannel junction using electrokinetic force, and separation was conducted with electrophoretic pullback. Sample was electrokinetically transported to and focused within the microchannel junction from the sample supply reservoir by applying 400 V to the sample waste reservoir 29 for 45 seconds, while grounding the remaining three reservoirs. The sample was then injected and separated in the first separation microchannel 26 by applying the following voltages: sample supply reservoir 28 and sample waste reservoir 29, 380 V, first separation microchannel reservoir 30, 700 V, and second separation microchannel reservoir 31, 0 V. Signal was collected by the first separation microchannel sensor 52 for 20 seconds. The resulting data are shown by profile “a” in FIG. 6A. Peaks bracketed by “1” are fluorescein, and those bracketed by “2” are FITC-peptide 1.

In a second experiment, pneumatic methods were used to transport sample to the microchannel junction from the sample supply reservoir and to confine sample within the junction. Electrophoretic pullback was used during separation. A partial vacuum was applied to the sample waste reservoir 29 for 6 seconds, then sample was injected and separated using the procedure and voltages described in Example 1. Signal was collected by the first separation microchannel sensor 52 for 20 seconds. The resulting data are shown in profile “b” of FIG. 6A.

In a third experiment, sample was transported electrokinetically from the sample supply reservoir 28 to the microchannel junction without sample focusing by applying 400 V to the sample waste reservoir 29, while grounding the sample supply reservoir 28 and allowing the first and second separation microchannel reservoirs 30 and 31 to float. After 12 seconds, sample was injected and separated in the first separation microchannel 26 without electrophoretic pullback by adjusting the voltages as follows: sample supply and sample waste reservoirs 28, 29 were allowed to float, first separation microchannel reservoir 30, 700 V, and second separation microchannel reservoir 31, 0 V. Signal was collected by the first separation microchannel sensor 52 for 20 seconds. Results from this experiment are shown by profile “c” in FIG. 6A.

In a fourth experiment, the effect of electrophoretic pullback on the peak shape and resolution of two anionic species was characterized after transporting sample to the microchannel junction with a pressure differential. Sample transport to and focusing within the microchannel junction was conducted pneumatically as described above. The focused sample stream was then injected into the first separation microchannel 26. Profile “b” represents separation conducted with pullback using the voltages given above for the second experiment. Profile “d” represents a separation conducted without pullback, using the following voltages: sample supply and sample waste reservoirs 28, 29 were allowed to float, first separation microchannel reservoir 30, 600 V, second separation microchannel reservoir 31, 0 V. Signal was collected by the first separation sensor 52 for 20 seconds. The results are shown in FIG. 6B.

### Example 3

Salt Effects on Sample Composition and Concentration Injected for Separations

Electrokinetic transport of charged species is subject to electrophoretic bias, which can significantly alter the composition and concentrations of charged species in a sample. This effect becomes more pronounced as the salt concentration of a sample increases because these ions carry an increasing fraction of the current. Sample transport using a pressure differential causes a bulk flow of fluids, and as a result sample composition is not altered. Experiments were conducted to quantitate the effects of salt on electrophoretic bias introduced by either electrokinetic or pneumatic sample transport. The sample for all of the experiments contained fluorescein and FITC-peptide 1 in 50 mM HEPES, plus either 0 mM, 25 mM, or 50 mM NaCl. The microchannels of the device were filled with separation buffer, as described in the general protocol preceding Example 1. Sample was introduced into the sample supply reservoir 28. For pneumatic experiments, sample was transported to the microchannel junction and separated as described in Example 1. For electrokinetic experiments, sample was transported and separated as described in the second paragraph of Example 2. Separations using electrokinetic transport are shown in FIG. 8A, and those using pneumatic transport are shown in FIG. 8B. Profiles “a” represent samples with no NaCl; profile “b” represents samples with 25 mM NaCl; profiles “c” represent samples with 50 mM NaCl. Peaks bracketed by “1” are fluorescein, and those bracketed by “2” are FITC-peptide 1.

For quantitation, the areas under the peaks of each bracketed series were integrated and divided by migration time. Values were then normalized relative to signal obtained from sample with no NaCl.

### Example 4

Simultaneous Separation and Detection of Both Cationic and Anionic Species

A bidirectional separation was conducted using pneumatic sample transport to the microchannel junction. Sample containing fluorescein, FITC-peptide 1, and FITC-peptide 2 was introduced to the sample supply reservoir 28. A vacuum of 4 psi was applied to the sample waste reservoir 29 for 10 seconds, while keeping the sample supply reservoir 28, and the first and second separation microchannel reservoirs 30 and 31 open to atmosphere. Injection of sample material into the first and second separation microchannels 26, 27 was done by breaking the vacuum, replenishing the sample waste reservoir by adding 8 μL separation buffer, and applying voltages as follows: sample supply and sample waste reservoirs 28, 29 were allowed to float, first separation microchannel reservoir 30, 1000 V, second separation microchannel reservoir 31, grounded. The first and second separation sensors 52, 53 collected signal for 20 seconds, generating two separate electropherograms. The results are shown in FIG. 9. The peaks correspond to sample species as follows: number 1, fluorescein; number 2, FITC-peptide 1; number 3, FITC-peptide 2. The electropherogram for the negative ions has been inverted in order to illustrate their migration from the point of injection relative to the positive ion.

All publications and patent applications mentioned in this specification are herein incorporated by reference to the same extent as if each individual publication or patent application was specifically and individually indicated as being incorporated by reference.

While illustrative embodiments have been chosen to provide details of the invention, it will be apparent to those of ordinary skill in the art that many changes and modifications can be made without departing from the spirit or scope of the invention as defined in the appended claims.

What is claimed is:

1. A method for separating charged sample components in a liquid sample comprising
introducing a liquid sample containing charged components into a first reservoir of a microfluidic device having a first microchannel extending between, and in fluid communication with, the first and a second reservoir, and a second channel extending between, and in fluid communication with, a third and a fourth reservoir, said two channels intersecting at a junction,
creating a pressure differential between the second reservoir and each of the first, third and fourth reservoirs, effective to move sample from the first reservoir through the junction, toward the second reservoir, and simultaneously, to move liquid contained in the separation channel, on opposite sides of said junction, toward the second reservoir, and
applying a potential difference between the third and fourth reservoirs, to produce electrophoretic separation of charged sample components present in the sample junction, as the charged components migrate from the junction toward the fourth reservoir in said second channel.

2. The method of claim 1, wherein said charged sample components including both positively and negatively charged species.

3. The method of claim 1, which further includes during said applying step, applying a potential difference between the first and the third reservoir, and between the second and the third reservoirs, effective to move charged species toward the first and second reservoirs from the separation channel in fluid communication with the third reservoir.

4. The method of claim 1, which further includes detecting one or more detectable characteristics of separated channel components, as they migrate in the separation channel toward the fourth reservoir.

5. A microfluidic system for separating charged sample components in a liquid sample comprising
a microfluidics device formed in a generally planar substrate and having (i) a first microchannel extending between, and in fluid communication with, a first and a second reservoir, and (ii) a second channel extending between, and in fluid communication with, a third and a fourth reservoir, said two channels intersecting at a junction,
a pressure-differential source operatively connected to said reservoirs for creating a pressure differential between the second reservoir and each of the first, third and fourth reservoirs, effective to move sample contained in the first reservoir through the junction, toward the second reservoir, and simultaneously, to move liquid contained in the separation channel, on opposite sides of said junction, toward the second reservoir, and
a voltage source operatively connected to said third and fourth reservoirs for applying a voltage potential between the third and fourth reservoirs, effective to produce electrophoretic separation of charged sample components present in the sample junction, as the charged components migrate from the junction toward the fourth reservoir in said second channel.

6. The system of claim 5, further comprising a control unit operatively connected to the pressure-differential source and the voltage source, for controlling the pressure differential and voltage potential applied to said reservoirs.

7. The system of claim 6, wherein the voltage source is operatively connected to the first and second reservoirs, for applying a voltage potential across the first and second reservoirs, while a voltage potential is applied across the third and fourth reservoirs, effective to move charged species toward the first and second reservoirs from the separation channel in fluid communication with the third reservoir, as sample components are moving in the separation channel towards the fourth reservoir.

8. The system of claim 5, wherein the first and second channels intersect to form a cross.

9. The system of claim 5, wherein the first channel includes a pair of arms that intersect the second channel at axially offset positions along the second channel, forming a region along a portion of the second channels between the intersections thereof with the two arms of the first channel.

10. The system of claim 5, wherein the first and second channels have widths and depths in the range of from about 1 μm to 200 μm.

11. The system of claim 10, wherein the first and second channels have widths and depths in the range of from about 30 μm to 80 μm.

12. The system of claim 5, wherein said channels have lengths in the range of 3 mm to 50 cm.

13. The system of claim 5, which further includes a detector positioned for analyzing characteristics of sample components moving within the second channel.

14. The system of claim 5, which further includes a pair of detectors positioned along the second channel on opposite sides of said junction, for analyzing characteristics of sample components moving within the second channel.
